(19) United States
(12) Patent Application Publication
(10) Pub. No.: US 2005/0282773 A1
(43) Pub. Date: Dec. 22, 2005

(54) Modified polysaccharides in combination with anti-cancer drugs for enhanced treatment of cancer

(75) Inventor: David Platt, Newton, MA (US)

Correspondence Address: PERKINS, SMITH & COHEN LLP, ONE BEACON STREET, 30TH FLOOR, BOSTON, MA, 02108, US

(73) Assignee: Pro-Pharmaceuticals, Inc., Newton, MA (US)

(21) Appl. No.: 11/182,096

(22) Filed: Jul. 15, 2005

(30) Foreign Application Priority Data
Jan. 14, 2004 (WO) PCT/US04/00747

(51) Int. Cl. A61K 31/715; C08B 37/00

(52) U.S. Cl. 514/54; 536/123

(57) ABSTRACT

Modified polysaccharide compositions and their use in combination with at least one anti-cancer drug for treating subjects with cancer, reduce toxicity and inhibit metastasis, are described. The modified polysaccharide includes a saccharide backbone being less than 5% esterified and containing repeating units, wherein each repeating unit has a plurality of uronic acid molecules, each repeating unit having at least one neutral monosaccharide attached thereto, at least one side chain of saccharides attached to the backbone further comprising a plurality of neutral saccharides or saccharide derivatives; and having an average molecule weight in the range of 15 to 60 kD. The polysaccharide when combine with the chemotherapeutic drug behave as a delivery vehicle, which positively enhance the chemotherapeutic effect while reducing side effects.

### RELATED APPLICATIONS

This application claims priority to and the benefit of U.S. provisional application Ser. No. 60/440,496, filed Jan. 16, 2003 and International application number PCT/US2004/000747, filed Jan. 14, 2004.

### TECHNICAL FIELD AND BACKGROUND ART

The present invention relates to chemically modified polysaccharides having a molecular weight in the range of 5 kD to 60 kD and a method of using the same in the delivery anticancer drugs for treating and preventing malignant cancer. It is known in the prior art that the incidence of many forms of cancer is expected to increase as the population ages. For example, prostate cancer is the most commonly diagnosed cancer in American men as well as the second leading cause of male cancer deaths. Approximately 50% of patients diagnosed with prostate cancer have a form of the disease which has or will escape the prostate. Prostate cancer metastasizes to the skeletal system and patients typically die with overwhelming osseous metastatic disease. As yet, effective curative therapy is limited and very little palliative therapy for patients with metastatic disease.

It is known in the prior art that the process of tumor cell metastasis requires that cells depart from the primary tumor, invade the basement membrane, traverse through the bloodstream from tumor cell emboli, interact with the vascular endothelium of the target organ, extravasate, and proliferate to form secondary tumor colonies, as described. Kohn, E., Anticancer Research, (1993), vol. 13, pp. 2553-2560 and Liotta, L. et al., Cell, (1991), vol. 64, pp. 327-336.

It is generally accepted that many stages of the metastatic cascade involve cellular interactions mediated by cell surface components such as carbohydrate-binding proteins, which include galactoside binding lectins (galectins) as described by Raz, A. et al., (1987) Cancer Metastasis Rev., vol. 6, p. 433; and Gabius, H.-J., Biochimica et Biophysica Acta, (1991), vol. 1071 pp 1-18. Treatment of B16 melanoma and uv-2237 fibrosarcoma cells in vitro with anti-galectin monoclonal antibodies prior to their intravenous (i.v.) injection into the tail vein of syngenic mice resulted in a marked inhibition of tumor lung colony development, as described by Meromsky, L. et al., Cancer Research, (1986), vol. 46, pp. 5270-5275. Transfection of low metastatic, low galectin-3 expressing uv-2237-c115 fibrosarcoma cells with galectin-3 cDNA resulted in an increase of the metastatic phenotype of the transfected cells, as described by Raz, A. et al., Int J. Cancer, (1990), vol. 46, pp. 871-877. Furthermore, a correlation has been established between the level of galectin-3 expression in human papillary thyroid carcinoma and tumor stage of human colorectal and gastric carcinomas, as described by Chiariotti, L. et al., Oncogene, (1992), vol. 7, pp. 2507-2511; Irimura, T. et al., Cancer Res., (1991), vol. 51, pp. 387-393; Lotan, R., et al., Int. J. Cancer, (1994), vol. 56, pp. 1-20; Lotz, M. et al., Proc. Natl. Acad. Sci., USA, (1993), vol. 90, pp. 3466-3470.

Simple sugars such as methyl-α-D-lactoside and lacto-N-tetrose have been shown to inhibit metastasis of B16 melanoma cells, while D-galactose and arabinogalactose inhibited liver metastasis of L-1 sarcoma cells, as described by Beuth, J. et al., J. Cancer Res. Clin. Oncol., (1987), vol. 113, pp. 51-55.

Most common proven anticancer drugs are cytotoxic due to non-specific delivery to the tumor site and side effects from toxicity to normal tissues. A desirable therapeutic combination would be where the polysaccharides reversibly trap the cytotoxic drugs, deliver them to the tumor site where the polysaccharides bind to the tumor and release the drug. This target delivery enhances therapeutic efficiency of these anticancer drugs, while substantially reduces their undesirable toxicity.

### SUMMARY OF THE INVENTION

The present invention relates to polysaccharide obtained by a chemical modification, which may reversibly interact with anti-cancer chemotherapeutic agent and effectively deliver it along with the polysaccharide itself to metastatic cancer, improving the pharmacological index as compared to that of the chemotherapeutic agent alone. In accordance with the present invention, the polysaccharide includes a saccharide backbone being less than about 5% esterified and containing repeating units wherein each repeating unit has a plurality of uronic acid or other glycosidic acid residues, with each repeating sequence unit having at least one neutral monosaccharide residue attached thereto; at least one side chain of oligosaccharide attached to the backbone via glucosidic bond to the neutral monosaccharide, further comprising a plurality of neutral saccharides or saccharide derivatives; with majority terminal galactose unit and an average molecular weight in the range of 5 to 60 kD. The polysaccharides 3 dimensional structure has a combination of hydrophobic, hydrophilic and slightly negative charged moieties which may interact with anti-cancer agents and effectively deliver them to metastatic cancer via interaction with galectin receptors. Thus the anticancer agents are being targeted to cancer cells effectively and initiate less toxicity to normal tissue.

Another embodiment of the present invention includes a polysaccharide as described above where the uronic acid saccharide backbone further includes galacturonic acid and the neutral monosaccharide connected to the repeating unit is a rhamnose. Another more specific embodiment provides a polysaccharide wherein at least one side chain comprises neutral saccharides and their derivatives connected to the backbone via rhamnose monosaccharides. This plurality in structure may enhance the polymer association with variety of anti-cancer drugs for more efficient delivery, while reducing side effects.

Yet other embodiments in accordance with the present invention include treating cancer in a subject diagnosed with cancer wherein a therapeutically effective amount of a known anti-cancer drug combined with a polysaccharide are co-administered to a subject. More specifically, the combination may be administered by any one of a plurality of routes including intravenous, subcutaneous, topical, intraperitoneal, and intramuscular routes. Another embodiment of the present invention is a method of preventing cancer in a subject post surgical intervention or diagnosed as having a high risk of cancer, wherein a therapeutically effective amount of a polysaccharide in combination with an anti-cancer drug is co-administered to a subject. More specifically, the polysaccharide and anti-cancer drug may be administered by any one of a plurality of routes including intravenous, subcutaneous, topical, intraperitoneal, and intramuscular routes. Still another embodiment of the present invention is a method for inhibiting metastasis in a subject wherein a therapeutically effective amount of a polysaccharide in combination with an anti-cancer drug is co-administered to the subject. More specifically, the polysaccharide and anti-cancer drug may be administered by any one of a plurality of routes including oral, intravenous, subcutaneous, topical, intraperitoneal, and intramuscular routes.

Yet other embodiments of the present invention include a pharmaceutical formulation for treating cancer wherein the formulation comprises an effective dose of a polysaccharide and a known anti-cancer drug, the polysaccharide having a backbone formed from a plurality of uronic acid saccharides and about one-in-seven to twenty-five neutral monosaccharides connected to the backbone, at least one side chain of neutral saccharides or saccharide derivatives connected via the neutral monosaccharide(s), and an average molecular weight in the range of 5 kD to 60 kD.

### DETAILED DESCRIPTION OF SPECIFIC EMBODIMENTS

The abbreviations used herein are: PS, polysaccharide; EHS, Eaglebreth-Holm Swarm; DMEM, Dulbecco's Modified Eagle's Minimal Essential Medium; CMF-PBS, Ca²⁺-and Mg²⁺-Free Phosphate-Buffered Saline, pH 7.2; BSA, Bovine Serum Albumin; galUA, galactopyranosyl uronic acid, also called galacturonic acid; and gal, galactose; man, mannose; glc, glucose; all, allose; alt, altrose; ido, idose; tal, talose; gul, gulose; and ara, arabinose, rib, ribose; lyx, lyxose; xyl, xylose; and fru, fructose; psi, psicose; sor, sorbose; tag, tagatose; and rha, rhamnose; fuc, fucose; quin, quinovose; 2-d-rib, 2-deoxy-ribose. As used in this description and the accompanying claims, the following terms shall have the meanings indicated, unless the context otherwise require:

“Administration” refers to oral, or parentereal including intravenous, subcutaneous, topical, transdermal, transmucosal, intraperitoneal, and intramuscular.

“Subject” refers to an animal such as a mammal for example a human.

“Treatment of cancer” refers to prognostic treatment of subjects at high risk of developing a cancer as well as subjects who have already developed a tumor. The term “treatment” may be applied to the reduction or prevention of abnormal cell proliferation, cell aggregation and cell dispersal (metastasis) to secondary sites.

“Cancer” refers to any neoplastic disorder, including such cellular disorders as, for example, renal cell cancer, Kaposi's sarcoma, chronic leukemia, breast cancer, sarcoma, ovarian carcinoma, rectal cancer, throat cancer, melanoma, colon cancer, bladder cancer, mastocytoma, lung cancer, mammary adenocarcinoma, pharyngeal squamous cell carcinoma, and gastrointestinal or stomach cancer.

“Depolymerization” refers to partial or complete hydrolysis of the polysaccharide backbone occurring for example when the polysaccharide is treated chemically resulting in fragments of reduced size when compared with the original polysaccharide.

“Effective dose” refers to a dose of an agent that improves the symptoms of the subject or the longevity of the subject suffering from or at high risk of suffering from cancer.

“Saccharide” refers to any simple carbohydrate including monosaccharides, monosaccharide derivatives, monosaccharide analogs, sugars, including those which form the individual units in an oligosaccharide or a polysaccharide.

“Monosaccharide” refers to polyhydroxyaldehyde (aldose) or polyhydroxyketone (ketose) and derivatives and analogs thereof.

“Oligosaccharide” refers to a linear or branched chain of monosaccharides that includes up to about 20 saccharide units linked via glycosidic bonds.

“Polysaccharide” refers to polymers formed from about 20 to about 10,000 and more saccharide units linked to each other by hemiacetal or glycosidic bonds. The polysaccharide may be either a straight chain, singly branched, or multiply branched wherein each branch may have additional secondary branches, and the monosaccharides may be standard D- or L-cyclic sugars in the pyranose (6-membered ring) or furanose (5-membered ring) forms such as D-frutose and D-galactose, respectively, or they may be cyclic sugar derivatives, for example amino sugars such as D-glucosamine, deoxy sugars such as D-fucose or L-rhamnose, sugar phosphates such as D-ribose-5-phosphate, sugar acids such as D-galacturonic acid, or multi-derivatized sugars such as N-acetyl-D-glucosamine, N-acetylneuraminic acid (sialic acid), or N-sulfato-D-glucosamine.

“Backbone” means the major chain of a polysaccharide, or the chain originating from the major chain of a starting polysaccharide, having saccharide moieties sequentially linked by either α or β glycosidic bonds. A backbone may comprise additional monosaccharide moieties connected thereto at various positions along the sequential chain.

“Esterification” refers to the presence of methylesters or other ester groups at the carboxylic acid position of the uronic acid moieties of a saccharide.

“Substantially de-esterified” means, for the purposes of this application, that the degree of esterification on the backbone of the polysaccharide is less than about 1% to 5%.

“Substantially lacks secondary branches of saccharides” means that the polysaccharide backbone has less than about 1-2 secondary branches per repeating unit.

In one embodiment of the invention, the modified polysaccharides have a uronic acid saccharide backbone or uronic ester saccharide backbones having neutral monosaccharides connected to the backbone about every one-in-twenty to every one-in-twenty-five backbone units. The resulting polysaccharides have at least one side chain comprised of mostly neutral saccharides and saccharide derivatives connected to the backbone via the about one-in-seven to twenty-five neutral monosaccharides. Some preferred polysaccharides may have at least one side chain of saccharides further having substantially no secondary saccharide branches, with a terminal saccharide comprising galactose, glucose, arabinose, or derivatives thereof. Other preferred polysaccharides may have at least one side chain of saccharides terminating with a saccharide modified by a feruloyl group.

Method of Making Modified Polysaccharides

In an embodiment of the invention, we prepared modified polysaccharides for use as co-therapeutic agents in treating cancer by a chemical modification of naturally occurring polymers. Prior to chemical modification, the polysaccharides may have a molecular weight range of between about 40,000-400,000 dalton with multiple branches of saccharides, for example, branches comprised of glucose, arabinose, galactose etc, and these branches may be connected to the backbone via neutral monosaccharides such as rhamnose. These molecules may further include a uronic acid saccharide backbone that may be esterified from as little as about 10% to as much as about 90% of uronic acid residues. The multiple branches themselves may have multiple branches of saccharides, the multiple branches optionally including neutral saccharides and neutral saccharide derivatives.

Described herewith is a chemical modification procedure that involves a pH-dependent depolymerization into smaller, de-branched polysaccharide molecules, using sequentially controlled pH, temperature and time e.g. pH 10.0 at 37 C for 30 minutes and than pH of about 3.5 at 25 C for 12 hours (see Example 1). An optional alternative modification procedure is hydrolysis of the polysaccharide in an alkaline solution in the presence of a reducing agent such as a potassium borohydride to form fragments of a size corresponding to a repeating subunit (U.S. Pat. No. 5,554,386). The molecular weight range for the chemically modified polysaccharides is in the range of 5 to 60 kD, more specifically, in the range of about 15-40 kD, and more specifically, for example, 20 kD.

Demonstrating the Biological Efficacy of Co-Administered Polysaccharides Along with Anti-Cancer Drugs Using In Vitro and In Vivo Assays

To show the efficacy of chemically modified polysaccharides co-administered in combination with known anti-cancer drugs, we have selected a number of in vitro and in vivo assays to demonstrate the biological efficacy of the compositions. Inhibition of metastasis can be shown using cancer cell lines which normally aggregate in culture, in the presence of the polysaccharide combined with the chemotherapy agent remain dispersed and more susceptible to the anti cancer drug. (Example 3 using B16-F1 cell, UV 2237-10-3 murine fibrosarcoma cells, HT 1080 human fibrosarcoma cells, and A375 human melanoma cells). Inhibition of metastasis can also be demonstrated using a Metastasis Assay (Example 4) in which MLL cells which have enhanced levels of galectins-3 on their cell surface, which is associated with tumor endothelial cell adhesion.

Vertebrate galactoside-binding lectins occur in a variety of tissues and cells. The lectins are divided into two abundant classes based on their sizes, with molecular masses about 14 kD and about 30 kD, and which are designated as galectin-1 and galectin-3, respectively. Galectin-3 represents a wide range of molecules i.e., the murine 34 kD (mL-34) and human 31 kD (hL-31) tumor-associated galactoside-binding lectins, the 35 kD fibroblast carbohydrate-binding protein (CBP35), the IgE-binding protein (cBP), the 32 kD macrophage non-integrin laminin-binding protein (Mac-2), and the rat, mouse, and human forms of the 29 kD galactoside-binding lectin (L-29). Molecular cloning studies have revealed that polypeptides of these lectins share identical aminoacid sequences.

Galectin-3 is highly expressed by activated macrophages and oncogenically transformed onto metastatic cells. Many cancer cells, including MLL cells, express galectin-3 on their cell surface and its expression has been implicated in metastatic processes in tumor cells. Elevated expression of the polypeptide is associated with an increased capacity for anchorage-independent growth, homotypic aggregation, and tumor cell lung colonization, which suggests that galectin-3 promotes tumor cell embolization in the circulation, and enhances metastasis. Tumor-endothelial cell adhesion is thought to be a key event in the metastatic process. Galectins may bind with high affinity to oligosaccharides containing poly-N-acetyllactosamine sequences, and also bind to the carbohydrate side chains of laminin in a specific sugar-dependent manner. Laminin, the major non-collagenous component of basement membranes, is an N-linked glycoprotein carrying poly-N-acetyllactosamine sequences, and is implicated in cell adhesion, migration, growth, differentiation, invasion and metastasis.

Tumor cells may interact with carbohydrate residues of glycoproteins via cell surface galectin-3 and this may be correlated with their ability to interact with the galactose residues of agarose (a polymer of D-galactose and L-anhydro-galactose) and to provide the minimal support needed for cell proliferation in this semi-solid medium. Anti-galectin-3 monoclonal antibodies can inhibit the growth of tumor cells in agarose. Furthermore, transfection of normal mouse fibroblasts with the mouse galectin-3 cDNA results in the acquisition of anchorage-independent growth. The in vivo results reported here with polysaccharides of example 1 are consistent with studies reported earlier and performed on human prostate cancer tissue using galectin-3 (U.S. Pat. No. 5,895,784). We propose that the polysaccharides described herein when combined with anti-cancer drugs for co-administration provide delivery, targeting and overall enhancement of anti-metastatic drugs in humans.

Co-Administration of a Polysaccharide and an Anti-Cancer Drug

The polysaccharides and anti-cancer drugs may be co-administered by any of several routes including oral, intravenous, subcutaneous, topical, intraperitoneal, and intramuscular routes, at equal intervals i.e., from about 10 to about 1000 mg/kg every 24 hours and/or from about 2.5 to 250 mg/kg every 6 hours.

Chemically modified polysaccharides and known anti-cancer drugs may be formulated for oral administration, either alone or together. Other routes of administration include topical, transdermal, intraperitoneal, intracranial, intracerebroventricular, intracerebral, intravaginal, intrauterine, oral, rectal or parenteral (e.g., intravenous, intraspinal, subcutaneous or intramuscular) route. In addition, the modified polysaccharide and the anti-cancer drug may be incorporated into biodegradable polymers allowing for sustained release of the compound, the polymers being implanted in the vicinity of where drug delivery is desired, for example, at the site of a tumor or implanted so that the modified polysaccharide is slowly released systemically. Osmotic mini-pumps may also be used to provide controlled delivery of high concentrations of modified polysaccharide through cannulae to the site of interest, such as directly into a metastatic growth or into the vascular supply to that tumor. The biodegradable polymers and their use are described, for example, in detail in Brem et al., J. Neurosurg., (1991), vol. 74, pp. 441-446.

The effective dose and dosage regimen of the polysaccharide and anti-cancer drug is a function of variables such as the subject's age, weight, medical history and other variables deemed to be relevant. The preferred dose and dosage regimen based on the molecular weight of the modified polysaccharide component (i.e., disregarding the digestible carrier), and similarly the anti-cancer drug, may include a daily dose of about 10 to about 1000 mg per kg of body weight of the subject. The dosages of the modified polysaccharide and anti-cancer drugs will depend on the disease state or condition being treated and other clinical factors such as weight and condition of the human or animal and the route of administration of the compound. Depending upon the half-life of the modified polysaccharide and the anti-cancer drug in the particular animal, either or both agents can be administered between several times per day to once a week. It is to be understood that the present invention has application for both human and veterinary use. The methods of the present invention contemplate single as well as multiple co-administrations, given either simultaneously or over an extended period of time.

Modified polysaccharide and anti-cancer drug formulations include those suitable for oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) administration. These formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit- dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, or an appropriate fraction thereof, of the administered ingredient. It should be understood that in addition to the ingredients, particularly mentioned above, the formulations of the present invention may include more than one agent as it is common in the field and have synergistic effect. Optionally, cytotoxic agents may be incorporated or otherwise combined with modified polysaccharides to provide dual therapy to the patient.

Suitable digestible pharmaceutical carriers include gelatin capsules in which the polysaccharide is encapsulated in dry form, or tablets in which polysaccharide is admixed with hydroxypropyl cellulose, hydroxypropyl methylcellulose, magnesium stearate, microcrystalline cellulose, propylene glycol, zinc stearate and titanium dioxide and other appropriate binding and additive agents. The composition may also be formulated as a liquid using distilled water, flavoring agents and some sort of sugar or sweetener as a digestible carrier to make a pleasant tasting composition when consumed by the subject.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The metastasis-modulating therapeutic composition of the present invention may be a solid, liquid or aerosol and may be administered by any known route of administration. Examples of solid therapeutic compositions include pills, creams, and implantable dosage units. The pills may be administered orally; the therapeutic creams may be administered topically. The implantable dosage units may be administered locally, for example at a tumor site, or which may be implanted for systemic release of the therapeutic angiogenesis-modulating composition, for example subcutaneously. Examples of liquid composition include formulations adapted for injection subcutaneously, intravenously, intra-arterially, and formulations for topical and intraocular administration. Examples of aerosol formulation include inhaler formulation for administration to the lungs.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit- dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, or an appropriate fraction thereof, of the administered ingredient. It should be understood that in addition to the ingredients, particularly mentioned above, the formulations of the present invention may include other agents conventional in the art having regard to the type of formulation in question. Optionally, cytotoxic agents may be incorporated or otherwise combined with angiostatin proteins, or biologically functional peptide fragments thereof, to provide dual therapy to the patient.

Other formulations for administering a therapeutic agent may be used that are well known in the art.

### EXAMPLES

### Example 1

Modifying Naturally Occurring Polysaccharides

A starting polysaccharide is sterilized by a treatment with U.V. radiation Or 70% alcohol for about 48 hours. All subsequent steps are conducted under sterile conditions. After sterilization the polysaccharide is slowly dissolved in distilled water. The amount of total carbohydrate is determined by the phenol sulfuric acid method (Fidler et al., Cancer Res., (1973), vol. 41, pp. 3642-3956.)

The pH of the polysaccharide solution is increased to pH 10.0 with, for example, using 3N NaOH. After appropriate time at 37° C., for example from 30 minutes to 24 hours, the solution is acidified with 3N HCl, for example, to a final pH of about 3.0 and maintained for example from 30 minutes to 24 hours, at 25° C. The solution is adjusted to a pH of about 6-7. Conditions are selected so the resulted modified polysaccharide has a molecular weight of 5 kD, 10 kD, 15 kD, 20 kD, 2.5 kD, 30 kD, 35 kD, 40 kD. The resulting modified polysaccharide product is washed with 70% ethanol and dried from 95% ethanol. Thereupon the modified polysaccharide is resolubilized in water to a final concentration of about 5-15% by weight (Alberscheim et al., Carbohydrate Research, (1967) vol. 5, pp. 340-346.) The modified polysaccharide may be further diluted for use according to embodiments of the invention in which concentrations of 0.01-5% may be provided to cells. Depending on the raw starting material and the desired modified polysaccharide composition and molecular weight, the reaction conditions can be further adjusted and modified accordingly.

### Example 2

Co-Administration of Polysaccharide and Anti-Cancer Drug to Animal with Tumors

A polysaccharide preparation is co-administered with a known cancer drug at a therapeutic dose of the known anti-cancer drug known in the art to be effective against cancer. As one example, 5-FU is administered at 2 to 20 mg/mL at doses of 60-600 mg/m²/day. As another example, paclitaxel is administered at 1-10 mg/mL at doses of 30 to 300 mg/m²/day. The best results in tumor bearing animals are seen when anticancer drugs, for example 5-FU and paclitaxel as described above, are combined with 2 to 20 mg/mL of a polysaccharide at doses of 60 to 600 mg/m². In particular, in rodents with colon tumors, up to 80% improvement was seen in the combination therapy versus the control with no treatment, and up to 40% improvement versus the 5-FU administered alone.

### Example 3

In Vitro Assays for Determining Anti-Cancer Efficacy of Mmodified Polysaccharides Co-Administered with Anti-Cancer Drugs.

(a) Unless stated otherwise, the assays below are conducted using a modified polysaccharide described in Example 1. Different sized molecules are tested ranging from 5 kD, 10 kD, 15 kD to 40 kD and including 20 kD, 2.5 kD, 30 kD, 35 kD prepared as described in Example 1. Amounts in w/v of modified polysaccharide vary from 0.01%-1% w/v in a physiological solution. Controls include polysaccharide alone with no additional agent, unmodified polysaccharide; gal-, ara- or feruloyl-substituted monosaccharide; and anti-cancer drug alone.
   Laminin and Asialoglycoprotein Adhesion Assays:

A good correlation has been established between the propensity of tumor cells to undergo homotypic aggregation in vitro and their metastatic potential in vivo. B16 melanoma cell clumps produce more lung colonies after i.v. injection than do single cells. Moreover, anti-galectin-3 antibody has been shown to inhibit asialofetuin-induced homotypic aggregation (Fidler, I. J., (1970) J. Natl. Cancer Inst., 45:77.), suggesting that the cell surface galectin-3 polypeptides bring about the formation of homotypic aggregates following their interaction with the side chains of glycoproteins.

A modified polysaccharide made according to Example 1 is tested for its ability, in the presence of an anti-cancer drug, to control cell-cell and cell-matrix interactions in B16-F1 murine melanoma cell adhesion assays which include measuring a change in adhesion of cells to a laminin coated substrate and inhibition of asialofetuin-induced homotypic aggregation and cell growth.

The B16-F1 line (low incidence of lung colonization) are derived from pulmonary metastasis produced by intravenous injection of B16 melanoma cells (Lotan, R. et al., Int. J. Cancer, (1994), vol. 56, pp. 1-20.) Other cell lines that can be tested include UV-2237-10-3 Murine Fibrosarcoma Cells, HT 1080 Human Fibrosarcoma Cells, and A375 Human Melanoma Cells.

The cells are grown in a monolayer on plastic in Dulbecco's modified Eagle's minimal essential medium (DMEM) supplemented with glutamine, essential amino acids, vitamins, antibiotics, and 10% heat-inactivated fetal bovine serum (FCS 10%). The cells are maintained at 37° C. in a humidified atmosphere of 7% CO₂, 93% air. To ensure reproducibility, all experiments should be performed with cultures grown for no longer than six weeks after recovery of stocks.

Laminin (EHS laminin) can be purchased from Sigma, St. Louis, Mo., and the modified polysaccharide in accordance with the present invention is prepared according to the above-described procedure. Asialofetuin can be prepared from fetuin, available from Gibco Laboratories. The fetuin is subjected to mild acid hydrolysis using 0.05 N H₂SO₄ at 80° C. for one hour, according to the method of Spire; Grand Island Biological Co., Grand Island. N.Y. (20). The released sialic acid is then removed from the fetuin by dialysis.

Cell Adhesion to Laminin

Tissue culture wells of 96-well plates are pre-coated overnight at 4° C. with EHS laminin (2 μg/well) in Ca²⁺-and Mg²⁺-free phosphate-buffered saline, pH 7.2 (CMF-PBS), and the remaining protein binding sites are blocked for 2 h at room temperature with 1% bovine serum albumin (BSA) in CMF-PBS. Cells are harvested with 0.02% EDTA in CMF-PBS and suspended with serum-free DMEM. A total of 5×10⁴ cells are added to each well in DMEM with or without: 1) modified polysaccharide and anti-cancer drug; 2) modified polysaccharides of varying concentrations with non-varying doses of anti-cancer drug; or 3) modified polysaccharide in a non-varying concentration with varying doses of anti-cancer drug. After incubation for 2 h 15 min at 37° C., non-adherent cells are washed off with CMF-PBS, and adherent cells are fixed with methanol and photographed. The relative number of adherent cells is determined in accordance with the procedure of Zollner, T. et al., Anti-cancer Research, (1993), vol. 13, pp. 923-930. Briefly, cells are stained with methylene blue followed by the addition of HCl-ethanol to release the dye. The optical density (650 ηm) is then measured by a plate reader.

Asialofetuin-Induced Homotypic Aggregation

Cells are detached with 0.02% EDTA in CMF-PBS and suspended at a concentration of 1×10⁶ cell/mL in CMF-PBS with or without 20 μg/mL of asialofetuin and 0% to 0.5% modified polysaccharide or 0% to 0.5% modified polysaccharide. Aliquots containing 0.5 mL of cell suspension are then placed in siliconized glass tubes and agitated at 80 rpm for 60 minutes at 37° C. The aggregation is then terminated by fixing the cells with 1% formaldehyde in CMF-PBS. Samples are used for counting the number of single cells, and the resulting aggregation is calculated according to the following equation: (1−Nₜ/N_{c})×100, where Nₜ and N_{c} represent the number of single cells in the presence of the tested compounds and that in the control buffer (CMF-PBS), respectively.

### Example 4

Modified Polysaccharide Binding to Galectin-3

Binding to Galactin has been correlated with membrane surface changes with changes in membrane permeability. Recombinant galectin-3 can be extracted from bacteria cells by single-step purification through an asialofetuin affinity column as described elsewhere. Recombinant galectin-3 eluted by lactose is extensively dialyzed against CMF-PBS before use. Horseradish peroxidase (HRP)-conjugated rabbit anti-rat IgG+IgM and the 2, 2′-azino-di(3-ethylbenzthiazoline sulfonic acid) (ABTS) substrate kit can be purchased from Zymed, South San Francisco, Calif. B16-F1 murine melanoma cells are grown as cultures in Dulbecco's modified Eagles' minimal essential medium (DMEM), as described above.

Tissue culture wells of 96-well plates are coated with CMF-PBS containing 0.5% MCP and 1% BSA and dried overnight. Recombinant galectin-3 serially diluted in CMF-PBS containing 0.5% BSA and 0.05% Tween-20 (solution A) in the presence or absence of 50 mM lactose is added and incubated for 120 minutes, after which the wells are drained and washed with CMF-PBS containing 0.1% BSA and 0.05% Tveen-20 (solution B). Rat antigalectin-3 in solution A is added and incubated for 60 minutes, followed by washing with solution B and incubation with HRP-conjugated rabbit anti-rat IgG+IgM in solution A for 30 minutes. After washing, relative amounts of bound enzyme conjugated in each well are ascertained by addition of ABTS. The extent of hydrolysis is measured at 405 ηn.

Colony Formation in Semi-Solid Medium

Cells are detached with 0.02% EDTA in CMF-PBS and suspended at 1×10³ cell/mL in complete DMEM with or without: 1) modified polysaccharide and anti-cancer drug; 2) modified polysaccharides of varying concentrations with non-varying doses of anti-cancer drug; or 3) modified polysaccharide in a non-varying concentration with varying doses of anti-cancer drug. The cells are incubated for 30 min at 37° C. and then mixed 1:1 (v/v) with a solution of 1% agarose in distilled water-complete DMEM (1:4, v/v) preheated at 45° C. Then, 2-μL aliquots of the mixture are placed on top of a pre-cast layer of 1% agarose in 6-cm diameter dishes. The cells are incubated for 14 days at 37° C., and the number of formed colonies is determined using an inverted phase microscope after fixation by the addition of 2.6% gluteraldehyde in CMF-PBS.

Competitive binding assays utilizing soluble recombinant galectin-3 and the anti-Mac-2 monoclonal antibodies can also be done, to compare their effects (or lack thereof) on cell adhesion to laminin, thereby providing some insight into how modified polysaccharide in combination with an anti-cancer drug may act in this regard.

Galectin-3 Heterotypic Aggregation Metastasis Assay

The MAT-LyLu (MLL) sub-line is a fast growing, poorly differentiated adenocarcinoma cell line. The adhesion of Cr-labeled MLL cells to confluent monolayers of rat aortic endothelial (RAE) cells in the presence or absence of modified polysaccharide is investigated. First, MLL and RAE cells are grown in RPMI 1640 media supplemented with 10% fetal bovine serum. RAE cells are grown to confluence in tissue culture wells. A total of 2.4×10⁶ MLL cells are incubated for 30 minutes with 5 μCi Na₅CrO₄ at 37° C., in 2 mL of serum-free media with 0.5% bovine serum albumin (BSA). Following extensive washing, 1×10³ MLL cells per well are added to RAE cell monolayers in quadruplicate. Attachment of MLL cells in the absence or presence of independently varied concentrations of combined modified polysaccharide and anti-cancer drug for 90 minutes at 4° C. is then assessed as follows. The cells are washed three times in cold phosphate-buffered saline to remove unbound cells, and then solubilized with 0.1 N NaOH for 30 minutes at 37° C., at which point the radioactivity in each well is determined in a beta-counter. The time course for the attachment of MLL cells to a confluent monolayer of RAE cells in the absence or presence of independently varied concentrations of modified polysaccharide in combination with an anti-cancer drug is monitored. The level of modified polysaccharide/anti-cancer drug inhibition on attachment of MLL cells to RAE cells is thereby determined.

Alternatively, in another variation of this assay, MLL cell adhesion to RAE cells is monitored through fluorescence methods. First, 1×10⁵ MLL cells are incubated for 30 minutes in 0.1% FITC to fluorescently label the cells. Following extensive washing the cells are added to RAE cell monolayers in 0.5% BSA. Independently varying concentrations of modified polysaccharide and anti-cancer drug combinations are added, for 30 or 60 minutes. The cultures are then washed to remove non-adherent cells, and the level of adhesion, or non-adhesion, is assessed based on fluorescence measurements.

To address the binding of modified polysaccharide to galectin-3, an enzyme-linked immunosorbent assay is employed to determine whether recombinant galectin-3 is able to bind immobilized modified polysaccharide in a dose-dependent manner, and whether the binding, if it occurs, is capable of being blocked by lactose. Results from such an assay allow assessment of the inhibitory effects on homotypic aggregation of a modified polysaccharide co-administered in combination with an anti-cancer drug, in accordance with the present invention, and determination of whether any modified polysaccharide binding occurs to cell surface galectin-3 molecules.

Semi-Solid Medium Metastasis-Mimic Assay

To determine the effect of modified polysaccharide in combination with an anti-cancer drug on MLL colony formation on 0.5% agarose, MLL cells are first detached from a cultured monolayer with 0.02% EDTA in Ca²⁺-and Mg²⁺-free (CMF)-PBS and suspended at a concentration of 4×10³ cells/mL in complete RPMI—with or without modified polysaccharide in varying concentrations. The cells are incubated for 30 minutes at 37° C., and them mixed 1:1 (v/v) with a solution of 1% agarose in RPMI 1:4 (v/v) which is preheated to 45° C. Next, 2-mL aliquots of the mixture are placed on top of a pre-cast layer of 1% agarose in 6-cm diameter dishes. The cells are incubated for 8 days at 37° C., fixed, counted and photographed. Phase contrast photomicrographs are prepared to show MLL cells grown without or with 0.1% (w/v) modified polysaccharide in combination with an anti-cancer drug.

Similar experiments can be done to investigate the effect of modified polysaccharide in combination with an anti-cancer drug on the rate of MLL cell growth in cultured monolayers in vitro, and the results can be compared to those obtained with in vivo experiments. In this way, information as to whether modified polysaccharide/anti-cancer drug co-treatment results in cytotoxicity can also be gained.

The ability of other tumor cells to form colonies in soft agar in the presence of modified polysaccharide and anti-cancer drug, including B16-F melanoma, UV-2237 fibrosarcoma, HT 1080 human fibrosarcoma, and A375 human melanoma, can also be investigated. The experiments would be carried out similarly to that described above for MLL cells.

### Example 5

In Vivo Assays for Determining Efficacy of Modified Polysaccharides in Combination with Anti-Cancer Drugs.

(a) Inhibition of Metastasis of R3327-MLL Cells in Vivo

The Dunning (R3327) rat prostate adenocarcinoma model of prostate cancer was developed by Dunning from a spontaneously occurring adenocarcinoma found in a male rat as described by Dunning, W., Natl. Cancer Inst. Mono., (1963), vol. 12, pp. 351-369. Several sub-lines have been developed from the primary tumor which have varying differentiation and metastatic properties as described by Isaacs, J. et al., Cancer Res., (1978), vol. 38, pp. 4353-4359. Injection of 1×10⁶ MLL cells into the thigh of the rat leads to animal death within approximately 25 days secondary to overwhelming primary tumor burden as described by Isaacs, J. et al., The Prostate, (1986), vol. 9, pp. 261-281, and Pienta, K., et al., The Prostate, (1992), vol. 20, pp. 233-241. The primary MLL tumor starts to metastasize approximately 12 days after tumor cell inoculation and removal of the primary tumor by limb amputation prior to this time results in animal cure. If amputation is performed after day 12, most of the animals die of lung and lymph node metastases within 40 days as described by Isaacs, J. et al., The Prostate, (1986), vol. 9, pp. 261-281.

Soluble modified polysaccharide, in combination with a known anti-cancer drug, is given orally to rats in the drinking water on a chronic basis, to investigate the affect on spontaneous metastases is these tumors. The rats are first injected with 1×10⁶ MLL cells in the hind limb on day 0. On day 4, when the primary tumors reach approximately 1 cm³ in size, 0.01%, 0.1%, or 1.0% (w:v) modified polysaccharide and anti-cancer drug is added to the drinking water of the rats on a continuous basis. On day 14, the rats are anesthetized and the primary tumors removed by amputating the hind limb. The rats are then followed to day 30 when all groups are sacrificed and autopsied. Animals continuously ingest modified polysaccharide/anti-cancer drug in their drinking water during this period. Control and treated animals are monitored for observable toxicity.

At day 30, the lungs are removed, rinsed in water and fixed overnight in Bouin's Solution. The number of rats which suffer lung metastases are compared to those in the control, and recorded. The number of MLL tumor colonies is determined by counting under a dissection microscope. The effect of modified polysaccharide/anti-cancer co-treatment is also monitored as a function of concentration in the drinking water. Throughout the study, treated animals are monitored for apparent toxicity and weight gain, and results are compared to the control group receiving no polysaccharide. Daily water intake is kept to 30±4 mL/rat in controls and treated groups. Hair texture, overall behavior, and stool color throughout the treatment period is also monitored and recorded for treated animals and control animals.

Control and co-treated animals will gain weight appropriately and no observable additional toxicity in the modified polysaccharide/anti-cancer treated animals is expected, compared to a control treatment of anti-cancer drug alone. The number of rats which suffer lung metastases will be reduced in animals fed with modified polysaccharides of the type described in example 1, in combination with a known anti-cancer drug, when compared with animals treated with the known anti-cancer drug alone, polysaccharide alone, individual monosaccharide residues or no polysaccharide or anti-cancer drug. A similar pattern of effect can be observed for lymph node disease. This example treatment is designed to show an improved method of treating an animal using non-toxic orally administered modified polysaccharide in combination with a known anti-cancer drug to prevent spontaneous cancer metastasis.

1. A modified polysaccharide combine with anti-cancer drug and comprising: a saccharide backbone being less than about 5% esterified and containing repeating units wherein each repeating unit has a plurality of uronic acid molecules, each repeating unit having at least one neutral monosaccharide attached thereto; at least one side chain of oligosaccharides attached to the backbone comprising a plurality of neutral oligosaccharides or oligosaccharide derivatives; and the modified polysaccharide having an average molecular weight in the range of 5 to 60 kD.

2. A modified polysaccharide according to claim 1, wherein the uronic acid saccharide of the backbone further comprise xylose, arabinose, ribose, lyxose, glucose, allose, altrose, idose, talose, galactose, gulose, mannose, fructose, psicose, sorbose, or tagatose.

3. A modified polysaccharide according to claim 1, wherein the uronic acid saccharides further comprise galacturonic acid.

4. A modified polysaccharide according to claim 1, wherein the neutral monosaccharides further comprise rhamnose.

5. A modified polysaccharide according to claim 1, wherein the average molecular weight of the polysaccharide is in the range of 5 to 60 kD.

6. A modified polysaccharide according to claim 1, wherein the average molecular weight of the polysaccharide is about 15 to 35 kD.

7. A modified polysaccharide according to claim 1, wherein the backbone is substantially de-esterified.

8. A modified polysaccharide according to claim 1, wherein the at least one oligosaccharide side chain is attached to the backbone via a neutral monosaccharide.

9. A modified polysaccharide according to claim 1, wherein the at least one oligosaccharide side chain is attached to the backbone via a rhamnose monosaccharide.

10. A modified polysaccharide according to claim 1, wherein the at least one oligosaccharide side chain further comprises galactose, mannose, glucose, allose, altrose, idose, talose, gulose, arabinose, ribose, lyxose, xylose, fructose, psicose, sorbose, tagatose, rhamnose, fucose, quinovose, 2-deoxy-ribose or their derivatives.

11. A modified polysaccharide according to claim 1, wherein the at least one oligosaccharide side chain terminates with a galactose, arabinose, rhamnose, glucose or derivatives thereof.

12. A modified polysaccharide according to claim 1, wherein the at least one oligosaccharide side chain terminates with a galactose.

13. A modified polysaccharide according to claim 1, wherein the at least one oligosaccharide side chain terminates with a feruloyl group.

14. A modified polysaccharide according to claim 1-13, wherein the at least one oligosaccharide side chain substantially lacks secondary branches of saccharides.

15. A modified polysaccharide according to claim 1-13, wherein the at least one oligosaccharide side chain has multiple secondary branches of saccharides.

16. A method of making a modified polysaccharide according to claim 1, comprising: selecting a composition having a saccharide backbone further comprising uronic acid saccharides and neutral monosaccharides and having between 5% and 95% esterification and having a plurality of side chains, at least one oligosaccharide side chain having secondary branching, the composition having an average molecular weight of between 45 kD and 400 kD; and performing a three-part chemical reaction consisting of depolymerizing the saccharide backbone, debranching the side chains; and de-esterifying the saccharide acid esters so as to make the modified polysaccharide.

17. A modified polysaccharide according to claim 16, wherein depolymerizing the composition is one part of the three-part chemical reaction, the depolymerizing further comprising treating the composition with an alkaline solution to provide a final pH of about 10.0.

18. A modified polysaccharide according to claim 17 wherein the debranching and de-esterifying occurs following the depolymerizing and further comprise treating the depolymerized composition with time temperature controlled reaction at a pH of about 10.0 and than treating with an acidic solution with time temperature controlled reaction at pH of about 3.0.

19. A modified polysaccharide combined with anti cancer drugs such as for example: Aminoglutethimide, Amsacrine Anastrozole, Asparaginase, Bicalutamide, Bleomycin, Buserelin, Busulfan, Capecitabine, Carboplatin, Carmustine, Chlorambucil, Cisplatin, Cladribine, Clodronate, Cyclophosphamide, Cyproterone, Cytarabine, Dacarbazine, Dactinomycin, Daunorubicin, dexamethasone, Diethylstilbestrol, Docetaxel, Doxorubicin, Epirubicin, Estramustine, Etoposide, Exemestane, Filgrastim, Fludarabine, Fludrocortisone, Fluorouracil, Fluoxymesterone, Flutamide, Gemcitabine, Goserelin, Hydroxyurea, Idarubicin, Ifosfamide, Imatinib, Interferon Alfa, Irinotecan, Letrozole, Leucovorin, Leuprolide, Levamisole, Lomustine, Mechlorethamine, Medroxyprogesterone, Megestrol, Melphalan, Mercaptopurine, Mesna, methamycins, Methotrexate, Mitomycin, Mitotane, Mitoxantrone, Nilutamide, Octreotide, Oxaliplatin, Paclitaxel, Pamidronate, Pentostatin, Plicamycin, Porfimer, Procarbazine, Raltitrexed, Rituximab, Streptozocin, Tamoxifen, Temozolomide, Teniposide, Testosterone, Thioguanine Thiotepa Topotecan Trastuzumab Tretinoin Vinblastine Vincristine Vindesine Vinorelbine, daunomycin, doxorubicin, vinblastine

20. The composition of claim 19 wherein said the taxine drug is selected from the group consisting of taxol; taxotere; spicatin; taxane-2, 13-dione, 5β, 9β, 10β-trihydroxy-,cyclic 9, 10-acetal with acetone, acetate; taxane-2, 13-dione, 5β, 9β, 10β-trihydroxy-trihydroxy-, cyclic 9, 10-acetal with acetone; taxane-2β, 5β, 9β, 10β-tetrol, cyclic 9, 10-acetal with acetone; taxane; cephalomannine-7-xyloside; 7-epi-10-deacetylcephalo-mannine; 10-deacetylcephalomannine; cephalomannine; taxol B; 13-(2′,3′-dihydroxy-3′-phenylpropionyl)baccatin III; yunnanxol; 7-(4-Azidobenzoyl)baccatin III; N-debenzoyltaxol A; O-acetylbaccatin IV; 7-(triethylsilyl)baccatin III; 7,10-Di-O-[(2,2,2-trichloroethoxy)carbonyl]baccatin III; baccatin III 13-0-acetate; baccatin diacetate; baccatin; baccatin VII; baccatin VI; baccatin IV; 7-epi-baccatin III; baccatin V; baccatin I; baccatin III; baccatin A; 10-deacetyl-7-epitaxol; epitaxol; 10-deacetyltaxol C; 7-xylosyl-10-deacetyltaxol; 10-deacetyltaxol-7-xyloside; 7-epi-10-deacetyltaxol; 10-deacetyltaxol; and 10-deacetyltaxol B.

21. A method for treating cancer in a subject, comprising: co-administering to a subject diagnosed with cancer, a therapeutically effective amount of a modified polysaccharide as described in claim 1 combined with an anti-cancer drug known to be effective for a specific cancer as describe in claim 19.

22. A method for treating cancer according to claim 19, wherein the cancer is renal cancer, sarcoma, Kaposi's sarcoma, chronic leukemia, breast cancer, mammary adenocarcinoma, ovarian carcinoma, rectal cancer, colon cancer, bladder cancer, prostrate cancer, melanoma, mastocytoma, lung cancer, throat cancer, pharyngeal squamous cell carcinoma, gastrointestinal cancer or stomach cancer.

23. A method for treating cancer according to claim 19, further comprising: co-administering the combined therapeutic amount of modified polysaccharide and anti-cancer drug to the subject by oral, intravenous, subcutaneous, topical, intraperitoneal, or intramuscular delivery routes.

24. A method for preventing cancer in a subject diagnosed as having a high risk of cancer, comprising: co-administering to a subject, a therapeutically effective amount of a modified polysaccharide combined with an anti-cancer drug as described in claim 1.

25. A method for preventing cancer according to claim 22, further comprising: co-administering the modified polysaccharide combined with an anti-cancer drug by oral, intravenous, subcutaneous, topical, intraperitoneal, intramuscular delivery routes or any combination of these routes.

26. A method for inhibiting metastasis in a subject, comprising: co-administering to a subject diagnosed with cancer, a therapeutically effective amount of a modified polysaccharide combined with an anti-cancer drug as described in claim 1.

27. A method for inhibiting metastasis according to claim 24, wherein the cancer is renal cancer, sarcoma, Kaposi's sarcoma, chronic leukemia, breast cancer, mammary adenocarcinoma, ovarian carcinoma, rectal cancer, colon cancer, bladder cancer, prostrate cancer, melanoma, mastocytoma, lung cancer, throat cancer, pharyngeal squamous cell carcinoma, gastrointestinal cancer or stomach cancer.

28. A method according to claim 24, further comprising: co-administering the modified polysaccharide and anti-cancer drug by oral, intravenous, subcutaneous, topical, intraperitoneal, intramuscular, or any combination of these routes.

29. A pharmaceutical formulation for treating cancer, comprising: an effective dose of a modified polysaccharide combined with an anti-cancer drug, the modified polysaccharide having a backbone formed from a plurality of uronic acid saccharides and about one-in-twenty neutral monosaccharides connected to the backbone, at least one side chain of neutral saccharides or saccharide derivatives connected via the neutral monosaccharide, an average molecular weight in the range of 5 kD to 60 kD, and; a pharmaceutically acceptable carrier.

30. A pharmaceutical formulation according to claim 29, wherein treating cancer further comprises inhibiting metastasis.

31. A pharmaceutical formulation according to claim 29, wherein the average molecular weight of the modified polysaccharide is in the range of 15 kD to 35 kD.

32. A pharmaceutical formulation according to claim 29, wherein the average molecular weight is about 25 kD.

33. A pharmaceutical formulation according to claim 29, wherein the uronic acid saccharides further comprise xylose, arabinose, ribose, lyxose, galactose, glucose, allose, altrose, idose, talose, gulose, mannose, fructose, psicose, sorbose, tagatose or derivatives thereof.

34. A pharmaceutical formulation according to claim 29, wherein the neutral monosaccharides include rhamnose.

35. A pharmaceutical formulation according to any one of claims 29-32, wherein the at least one oligosaccharide side chain substantially lacks secondary branches of saccharides.

36. A pharmaceutical formulation according to any one of claims 29-32, wherein the at least one oligosaccharide side chain has a plurality of secondary branches of saccharides.
